(19) United States
(12) Patent Application Publication
(10) Pub. No.: US 2006/0062734 A1
(43) Pub. Date: Mar. 23, 2006

(54) Methods and systems for preventing diversion of prescription drugs

(76) Inventors: Richard J. Melker, Gainesville, FL (US); Mark S. Gold, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/945,732

(22) Filed: Sep. 20, 2004

(51) Int. Cl. A61K 49/00

(52) U.S. Cl. 424/10.1

(57) ABSTRACT

The subject invention provides systems and methods for monitoring prescription medications. In particular, systems and methods are provided for monitoring patient compliance with a given prescribed regimen as well as monitoring the origins of a prescription drug. The subject invention provides a central computer and a portable device, wherein the portable device includes at least one sensor for detecting a target marker. The target marker of the invention represents either the presence of a specific prescribed medication or identify the proper origins of a medication.

### BACKGROUND OF THE INVENTION

Counterfeit drugs are a serious public health and safety concern. If introduced to a drug supply, counterfeit drugs can pose significant health risks to thousands, if not millions, of people including: toxic effects, unintended effects, and ineffective treatments. Since counterfeit drugs can contain either only inactive ingredients, incorrect ingredients, improper dosages, or even dangerous sub-potent or super-potent ingredients, patients face the risk of therapeutic failures, at the least, and worsening of health problems, perhaps even leading to fatal consequences.

Distribution of legitimate pharmaceuticals is dependent on the wholesale industry. Primary wholesalers purchase drugs directly from manufacturers and then sell the products directly to a pharmacy, hospital, institution, other dispenser, or secondary wholesaler. In the U.S., three primary wholesalers account for 90% of distributed prescription drugs. Occasionally when low-cost drugs are available (for example, because of temporary excess in the supply of a drug), primary wholesalers purchase from secondary wholesalers. Secondary wholesalers usually deal in smaller quantities and have higher turnover of stock. But in some instances, some smaller wholesalers also knowingly or unknowingly take higher risks by obtaining drugs that may not have been provided by a legitimate manufacturer. Thus, counterfeit drugs can enter a drug distribution supply chain via the secondary wholesale market, where drugs can change hands several times before reaching the end user.

For example, unlicensed or unregulated pharmacies may knowingly or unknowingly distribute unapproved drugs. In addition, counterfeit drugs can enter the market via disguised imports from other countries (for example, gray market goods), or through the purchase of drugs via the Internet.

Another serious problem is the diversion of licit drugs for illicit purposes (also known as prescription drug diversion). The United States Drug Enforcement Agency reports that prescription drug diversion accounts for about 30% of the overall drug problem in the United States. As opposed to other commonly abused drugs (such as, marijuana, heroin), prescription drugs can be obtained through legal channels. These drugs are attractive to substance abusers because they are manufactured legitimately and prescribed by physicians, giving them the illusion of safety. In certain instances, the addiction and withdrawal associated with the abuse of many prescription drugs can be more harmful than that associated with illegal drugs.

The most commonly diverted pharmaceutical drugs include: opioids (such as, OxyContin, Darvon, Vicodin, Dilaudid, Demerol, and Lomotil); cerebral nervous system depressants (such as, Mebaral, Nembutal, Valium, Librium, Xanax, Halcion, and ProSom); and stimulants (such as, Dexedrine, Ritalin, and Meridia). Although such pharmaceutical drugs have legitimate medical purposes, they are often illegally diverted for recreational use, which costs the federal government and states billions of dollars in areas such as law enforcement, health care, social services, and court costs.

The diversion of legitimate prescription drugs typically occurs through: (1) doctor shopping; (2) illegal internet pharmacies; (3) drug theft; (4) prescription forgery; and (5) illicit prescriptions by physicians. Doctor shopping is one of the most popular methods of obtaining prescription drugs for illegal use. It typically involves an individual obtaining a wide array of prescriptions and, rather than taking the drugs as prescribed, selling them illegally (see Pilar Kraman, “Drug Abuse in America—Prescription Drug Diversion,” Trends Alert, The Council of State Governments (April 2004)).

For example, narcotics and benzodiazepines are prescribed in high doses to patients who experience a great deal of pain (for example, patients diagnosed with cancer and other conditions that result in chronic, unremitting pain). Because such patients often become tolerant to these drugs due to protracted course of their disease, escalated dosages of the drugs are required to control their pain. These individuals are often incapacitated, and it is relatively easy for family members, caretakers, and others to divert a portion of the prescribed medication for illegal sale or use.

Current methods for ensuring the availability of prescription medications for legitimate medical conditions while preventing their diversion to the illegal market include: (1) prescription monitoring programs; (2) drug education for health care professionals; and (3) theft/fraud regulation. Current prescription drug monitoring programs involve either the use of multiple prescriptions or electronic transmission.

Multiple prescription programs require physicians to use multiple-copy, state-issued prescription pads that contain serial numbers. One copy is sent to the state regulatory agency after the prescription is filled. Electronic transmission programs are based on the multiple prescription program; such programs require the pharmacist to transmit prescription information via the computer to the designated state agency. Unfortunately, these programs may affect patient care when doctors hesitate or cease to prescribe certain regulated drugs. Moreover, neither physicians nor pharmacists have any means for monitoring patient compliance with a prescribed regimen. Finally, such monitoring programs do not enable capture of counterfeit drugs.

Therefore, there is a need for effective, user-friendly systems that can monitor patient compliance with a prescribed regimen, as well as ensure legitimate drug distribution to a patient from a pharmacy.

### BRIEF SUMMARY

One objective of the subject invention is to provide systems and methods for monitoring patient compliance in taking a medication as prescribed. Another objective of the invention is to provide systems and methods for verifying the origins of a medication. Accordingly, the system of the invention comprises a central computer and a portable device equipped with at least one sensor specific for a marker. For example, a portable device of the invention can be provided with at least one sensor specific for a marker that is representative of a prescribed medication and/or at least one sensor specific for a marker that represents the medication's proper origins.

In certain embodiments of the invention, the portable device includes any number of known identification systems such as fingerprinting or retinal scanning technology. The portable device of the invention can also include, without limitation, a means for receiving a sample (for example, from a patient and/or headspace from a prescription medication container) and a processing means. Preferably, the portable device can detect a marker of a specifically prescribed medication in exhaled breath. The processing means includes a means for receiving data provided via the sensor(s) and a means for determining whether an action/event (such as, a biological sample that has been provided to the portable device of the invention) has occurred within a configurable time interval.

The processing means of the portable device can also contain wireless or standard communication technology to automatically relay information to a pharmacist or other monitoring personnel regarding whether an action has occurred (for example, a medication is being taken as prescribed). Alternatively, the portable device can include a means for storing the data from the sensor, where the data can be downloaded to a central computer of the invention at the time the prescription is refilled.

The central computer of the invention can include: (1) a means for tracking dispensed medications and corresponding prescription; (2) a means for receiving input from the portable device regarding whether the medication is being taken as prescribed; and (3) a means for notifying the pharmacist or other system monitoring personnel that the medication is or is not being taken as prescribed.

In one method of use, a patient will be provided with a prescribed medication and a portable device from the pharmacist. At a specified time interval after each dosage of the medication, the patient will provide a sample of bodily fluid to the device. Preferably, the patient exhales into the device. According to the subject invention, the sample of bodily fluid will be applied to the sensor(s) of the portable device. Detection of a target marker provides notice of the medication's presence in the patient and consequently, allows assessment of whether a drug has been taken as prescribed. In another embodiment, the concentration of the target marker in the sample of bodily fluid can be quantified.

Information from the sensor is then processed by a processing means within the portable device, which can then be provided to the central computer of the invention to document that the medication is being taken by the patient, and also that previous doses were taken as prescribed, based on the concentration of the target marker in a sample of bodily fluid (such as, exhaled breath).

In another method of use, a medication will be manufactured with a particular volatile marker (or “taggant”) for use in detecting counterfeit medications. Specifically, the taggant from the medication will be detectable in the headspace of a medication container. At the time a pharmacist initially opens a bottle of medication, the headspace of the bottle would be sampled with a portable device of the invention to detect the taggant. If the sensor(s) of the portable device does not detect the proper taggant (or even the proper taggant at the appropriate concentrations), the pharmacist would know that the medication is counterfeit, isolate and prevent medication distribution, and notify the proper authorities of the alleged counterfeit drug.

In a related embodiment, medication containers can be manufactured that contain a taggant for use in identifying whether the drug within is the original drug produced by the manufacturer. For example, a medication container can be produced with a taggant (such as, in the cap, within the interior of the container) that would be readily detected using a portable device of the invention. Alternatively, packaging items (such as, cotton fillers, desiccants, etc.) can be manufactured with a taggant and then placed in the medication container for use in identifying counterfeit drugs.

In a method of use, a pharmacist will be provided with information regarding the taggant that should be present in a medication container. Information regarding the taggant can be provided to the pharmacist using any known communication methods including, for example, on a coded invoice; a scannable bar-code on the medication container; facsimile, voice message, electronic message, or postal message. Where necessary, certain embodiments include secure communication methods including, for example, encrypted internet communications. When the pharmacist opens the medication container, the headspace of the container would be sampled using the portable device of the invention. If the sensor(s) of the portable device does not detect the proper taggant (and in certain instances, the proper taggant concentration), the pharmacist would know that the medication is counterfeit, isolate and prevent medication distribution, and notify the proper authorities of the alleged counterfeit drug.

In accordance with the present invention, a sensor of the invention comprises well-known biodetectors or biosensors. Commonly available biodetectors or biosensors are based upon naturally occurring and/or synthetic compounds having high specificity and sensitivity to chemical and/or biological compounds of interest (such as a marker of the invention). Suitable biodetectors or biosensors of the invention include, but are not limited to, those based on antibodies, enzymes, proteins, receptors, peptides, nucleic acids, membranes, whole and/or living cells, and aptamers.

The advantages of the invention are numerous. First and foremost, for healthcare personnel, the invention provides a method that can readily assess (for example, point-of-care assessment) whether a patient has followed the course of a prescribed medication based on a sample of the patient's bodily fluid. Second, the invention is inexpensive and has broad medical applications (for example, more accurate medical treatment where physicians can readily assess the effectiveness of a treatment regimen). Further, the invention can be useful for law enforcement/public health and safety purposes (such as in confiscating counterfeit and/or diverted prescription drugs).

### DESCRIPTION OF THE DRAWINGS

FIGS. 1-4 show embodiments of suitable flowcharts of the functional steps performed by a portable device of the invention in implementing the measurement and analysis procedures for a sample.

FIG. 5 is a computing means used in accordance with the subject invention.

### DETAILED DISCLOSURE

The present invention is broadly directed to the efficient, timely, and accurate monitoring of prescription drugs. In certain embodiments of the invention, systems and methods are provided for analyzing a sample of a patient's bodily fluids to assess whether the patient is adhering to a prescribed regimen and to track dispensed medications. In other embodiments, the origin of a prescription drug can be determined using the systems and methods of the invention.

The systems and methods of the invention are based on the use of commonly available sensor and computer technology. According to the subject invention, sensor technology is applied to a sample of bodily fluid and/or to headspace of a prescription drug container to detect the presence of target marker(s). Information regarding the target marker(s) is used to: (1) track dispensed medications; (2) monitor patient compliance in adhering to a prescribed regimen; and (3) monitor the origins of a prescription drug to ensure the drug is not a counterfeit or diverted prescription drug.

Definitions

Unless otherwise stated, the following terms used in the specification and claims have the meanings given below.

The term “bodily fluid,” as used herein, refers to a mixture of molecules obtained from a patient. Bodily fluids include, but are not limited to, exhaled breath, whole blood, blood plasma, urine, semen, saliva, lymph fluid, meningal fluid, amniotic fluid, vaginal fluid, glandular fluid, sputum, feces, sweat, mucous, and cerebrospinal fluid. Bodily fluid also includes experimentally separated fractions of all of the preceding solutions or mixtures containing homogenized solid material, such as feces, tissues, and biopsy samples.

The term “marker,” as used herein, refers to a molecule or compound that is detectable by means of its physical or chemical properties. According to the subject invention, a marker can be the medication itself, metabolites of the medication, endogenous by-products produced in metabolizing the medication, or volatile markers. In certain embodiments, volatile markers are attached to medication, where the volatile markers are released after the medication is metabolized. In other embodiments, a particular volatile marker or “taggant” is added to a prescription drug container for use in confirming the origin of the drug.

A “patient,” as used herein, describes an organism, including mammals, from which bodily fluid samples are collected in accordance with the present invention. Mammalian species that benefit from the disclosed systems and methods of diagnosis include, and are not limited to, apes, chimpanzees, orangutans, humans, monkeys; and domesticated animals (e.g., pets) such as dogs, cats, mice, rats, guinea pigs, and hamsters.

Sensor Technology

Sensor technology is used by the present invention to detect the presence of a marker in a bodily fluid sample and/or in headspace of a prescription drug container. Sensors contemplated for use with the systems and methods of the invention include, but are not limited to, immunoassay, immunosensor, and biosensor technology.

Immunoassay and immunosensor technology are based on the specificity of molecular recognition by complexation agents (such as antibodies, aptamers, proteins, or molecular imprinted polymers) to form a stable complex in solution for the immunoassay and on solid-state interfaces for the immunosensor. For both technologies, the specificity for the measurement of a marker as well as the expression of the stable complex are dependent on the application of the complexation agent. New developments in protein engineering for immunoglobulins (including antibody fragments and chimeric antibodies); in substituting antibodies by alternative binding components (such as aptamers) or structures (such as molecular imprinting); and in coupling fusion proteins to reporter molecules will, therefore be applicable to either immunosensor or immunoassay technology, if available.

Biosensor technology is based on the integration of a biological element on a solid-state surface for biospecific interaction with a target marker. The biological element can include any molecule qualified for biorecognition including, but not limited to, enzymes, receptors, peptides, lectins, specific binding proteins, nucleic acids including single-stranded DNA, membranes, and living cells. In certain instances, biosensor technology and immunosensor technology overlap. For example, a biological element can include antibodies or antibody-related substances.

There are several sensor devices available based on immunoassay, imunosensor, or biosensor technology. They include, without limitation, surface acoustic wave (SAW) sensors (such as those disclosed in U.S. Pat. Nos. 4,312,228 and 4,895,017, and Groves W. A. et al., “Analyzing organic vapors in exhaled breath using surface acoustic wave sensor array with preconcentration: Selection and characterization of the preconcentrator. adsorbent,” Analytica Chimica Acta, 371:131-143 (1988)); quartz microbalance sensors, metal oxide sensors, chemical sensors known in the art that use chemoselective coating applicable to the operation of the present invention (such as bulk acoustic wave (BAW) devices, plate acoustic wave devices, interdigitated microelectrode (IME) devices, optical waveguide (OW) devices, electrochemical sensors, and electrically conducting sensors); fluid sensor technology (such as commercial devices known as “artificial noses,” “electronic noses,” or “electronic tongues” or as disclosed in U.S. Pat. Nos. 5,945,069; 5,918,257; 5,891,398; 5,830,412; 5,783,154; 5,756,879; 5,605,612; 5,252,292; 5,145,645; 5,071,770; 5,034,192; 4,938,928; and 4,992,244; and U.S. Patent Application No. 2001/0050228); semiconductive gas sensors; mass spectrometers; IR, UV, visible, or fluorescence spectrophotometers; and apparatuses having conductive-polymer gas-sensors (“polymeric”), aptamer biosensors, amplifying fluorescent polymer (AFP) sensors, and microcantilever sensors. The following are examples of various sensor technologies that may be utilized in practicing the method of the present invention:

Microgravimetric Sensors

Microgravimentric sensors are based on the preparation of polymeric- or biomolecule-based sorbents that are selectively predetermined for a particular substance, or group of structural analogs. A direct measurement of mass changes induced by binding of a sorbent with a target marker can be observed by the propagation of acoustic shear waves in the substrate of the sensor. Phase and velocity of the acoustic wave are influenced by the specific adsorption of target markers onto the sensor surface. Piezoelectric materials, such as quartz (SiO₂) or zinc oxide (ZnO), resonate mechanically at a specific ultrasonic frequency when excited in an oscillating field. Electromagnetic energy is converted into acoustic energy, whereby piezoelectricity is associated with the electrical polarization of materials with anisotropic crystal structure. Generally, the oscillation method is used to monitor acoustic wave operation. Specifically, the oscillation method measures the series resonant frequency of the resonating sensor. Types of sensors derived from microgravimetric sensors include quartz crystal microbalance (QCM) devices that apply a thickness-shear mode (TSM) and devices that apply surface acoustic wave (SAW) detection principle. Additional devices derived from microgravimetric sensors include the flexural plate wave (FPW), the shear horizontal acoustic plate (SH-APM), the surface transverse wave (STW) and the thin-rod acoustic wave (TRAW).

Surface Acoustic Wave Sensors (SAW)

SAW sensors are constructed with electrodes that generate and detect surface acoustic waves based on surface activity. Surface acoustic waves are waves that have their maximum amplitude at the surface and whose energy is nearly all contained within 15 to 20 wavelengths of the surface. Because the amplitude is a maximum at the surface such devices are very surface sensitive.

SAW chemical sensors take advantage of this surface sensitivity to function as sensors. To increase specificity for specific compounds, SAW devices are frequently coated with a thin polymer film that will affect the frequency and insertion loss of the device in a predictable and reproducible manner. Each sensor in a sensor array is coated with a different polymer and the number and type of polymer coating are selected based on the chemical to be detected. If the device with the polymer coating is then subjected to chemical vapors that absorb into the polymer material, then the frequency and insertion loss of the device will further change. It is this final change that allows the device to function as a chemical sensor.

If several SAW devices are each coated with a different polymer material, the response to a given chemical vapor will vary from device to device. The polymer films are normally chosen so that each will have a different chemical affinity for a variety of organic chemical classes, that is, hydrocarbon, alcohol, ketone, oxygenated, chlorinated, and nitrogenated. If the polymer films are properly chosen, each chemical vapor of interest will have a unique overall effect on the set of devices. SAW chemical sensors are useful in the range of organic compounds from hexane on the light, volatile extreme to semi-volatile compounds on the heavy, low volatility extreme.

Motors, pumps and valves are used to bring the sample into and through the array. The sensitivity of the SAW system can be enhanced for low vapor concentrations by having the option of using a chemical preconcentrator before the array. In operation, the preconcentrator absorbs the test vapors for a period of time and is then heated to release the vapors over a much shorter time span thereby increasing the effective concentration of the vapor at the array. The SAW system uses some type of drive and detection electronics for the array. An on board microprocessor is used to control the sequences of the SAW system and provide the computational power to interpret and analyze data from the array.

SAW sensors are reasonably priced (less than $200) and have good sensitivity (tens of ppm) with very good selectivity. They are portable, robust and consume nominal power. They warm up in less than two minutes and require less than one minute for most analysis. They are typically not used in high accuracy quantitative applications, and thus require no calibration. SAW sensors do not drift over time, have a long operating life (greater than five years) and have no known shelf life issues. They are sensitive to moisture, but this is addressed with the use of a thermally desorbed concentrator and processing algorithms.

Thickness-Shear Mode Sensors (TSM)

TSM sensors consist of an AT-cut piezoelectric crystal disc, most commonly of quartz because of its chemical stability in biological fluids and resistance to extreme temperatures, and two electrodes (preferably metal) attached to opposite sides of the disc. The electrodes apply the oscillating electric field. Generally, TSM sensor devices are run in a range of 5-20 MHz. Advantages are, besides the chemical inertness, the low cost of the devices and the reliable quality of the mass-produced quartz discs.

Conducting Polymers

Conducting polymer sensors promise fast response time, low cost, and good sensitivity and selectivity. The technology is relatively simple in concept. A conductive material, such as carbon, is homogeneously blended in a specific non-conducting polymer and deposited as a thin film on an aluminum oxide substrate. The films lie across two electrical leads, creating a chemoresistor. As the polymer is subjected to various chemical vapors, it expands, increasing the distance between carbon particles, and thereby increasing the resistance. The polymer matrix swells because analyte vapor absorbs into the film to an extent determined by the partition coefficient of the analyte. The partition coefficient defines the equilibrium distribution of an analyte between the vapor phase and the condensed phase at a specified temperature. Each individual detector element requires a minimum absorbed amount of analyte to cause a response noticeable above the baseline noise. Selectivity to different vapors is accomplished by changing the chemical composition of the polymer. This allows each sensor to be tailored to specific chemical vapors. Therefore, for most applications an array of orthogonal responding sensors is required to improve selectivity. Regardless of the number of sensors in the array, the information from them must be processed with pattern recognition software to correctly identify the chemical vapors of interest. Sensitivity concentration are reportedly good (tens of ppm). The technology is very portable (small and low power consumption), relatively fast in response time (less than 1 minute), low cost, and should be rugged and reliable

Electrochemical Sensors

Electrochemical sensors measure a change in output voltage of a sensing element caused by chemical interaction of a target marker on the sensing element. Certain electrochemical sensors are based on a transducer principle. For example, certain electrochemical sensors use ion-selective electrodes that include ion-selective membranes, which generate a charge separation between the sample and the sensor surface. Other electrochemical sensors use an electrode by itself as the surface as the complexation agent, where a change in the electrode potential relates to the concentration of the target marker. Further examples of electrochemical sensors are based on semiconductor technology for monitoring charges at the surface of an electrode that has been built up on a metal gate between the so-called source and drain electrodes. The surface potential varies with the target marker concentration.

Additional electrochemical sensor devices include amperometric, conductometric, and capacitive immunosensors. Amperometric immunosensors are designed to measure a current flow generated by an electrochemical reaction at a constant voltage. Generally, electrochemically active labels directly, or as products of an enzymatic reaction, are needed for an electrochemical reaction of a target marker at a sensing electrode. Any number of commonly available electrodes can be used in amperometric immunosensors, including oxygen and H₂O₂ electrodes.

Capacitive immunosensors are sensor-based transducers that measure the alteration of the electrical conductivity in a solution at a constant voltage, where alterations in conductivity are caused by biochemical enzymatic reactions, which specifically generate or consume ions. Capacitance changes are measured using an electrochemical system, in which a bioactive element is immobilized onto a pair of metal electrodes, such as gold or platinum electrodes.

Conductometric immunosensors are also sensor-based transducers that measure alteration of surface conductivity. As with capacitive immunosensors, bioactive elements are immobilized on the surface of electrodes. When the bioactive element interacts with a target marker, it causes a decrease in the conductivity between the electrodes.

Electrochemical sensors are excellent for detecting low parts-per-million concentrations. They are also rugged, draw little power, linear and do not require significant support electronics or vapor handling (pumps, valves, etc.) They are moderate in cost ($50 to $200 in low volumes) and small in size.

Gas Chromatography/Mass Spectroscopy (GC/MS)

Gas Chromatography/Mass Spectroscopy (GC/MS) is actually a combination of two technologies. One technology separates the chemical components (GC) while the other one detects them (MS). Technically, gas chromatography is the physical separation of two or more compounds based on their differential distribution between two phases, the mobile phase and stationary phase. The mobile phase is a carrier gas that moves a vaporized sample through a column coated with a stationary phase where separation takes place. When a separated sample component elutes from the column, a detector converts the column eluent to an electrical signal that is measured and recorded. The signal is recorded as a peak in the chromatogram plot. Chromatograph peaks can be identified from their corresponding retention times. The retention time is measured from the time of sample injection to the time of the peak maximum, and is unaffected by the presence of other sample components. Retention times can range from seconds to hours, depending on the column selected and the component. The height of the peak relates to the concentration of a component in the sample mixture.

After separation, the chemical components need to be detected. Mass spectroscopy is one such detection method, which bombards the separated sample component molecules with an electron beam as they elute from the column. This causes the molecules to lose an electron and form ions with a positive charge. Some of the bonds holding the molecule together are broken in the process, and the resulting fragments may rearrange or break up further to form more stable fragments. A given compound will ionize, fragment, and rearrange reproducibly under a given set of conditions. This makes identification of the molecules possible. A mass spectrum is a plot showing the mass/charge ratio versus abundance data for ions from the sample molecule and its fragments. This ratio is normally equal to the mass for that fragment. The largest peak in the spectrum is the base peak. The GC/MS is accurate, selective and sensitive.

Optical Sensors

Optical sensors are based on the application of visible radiation for use in rapid signal generation and reading. For example, changes in adsorption, fluorescence, luminescence, scatter or refractive index (RI) are all useful occurrences when light is reflected at sensing surfaces for use in detecting a target marker.

Infrared (IR) spectroscopy is one of the most common spectroscopic techniques used by organic and inorganic chemists. Simply, it is the absorption measurement of different IR frequencies by a sample positioned in the path of an IR beam. IR radiation spans a wide section of the electromagnetic spectrum having wavelengths from 0.78 to 1000 micrometers (microns). Generally, IR absorption is represented by its wave number, which is the inverse of its wavelength times 10,000. For a given sample to be detected using IR spectroscopy, the sample molecule must be active in the. IR region, meaning that the molecule must vibrate when exposed to IR radiation. Several reference books are available which contain this data, including the Handbook of Chemistry and. Physics from the CRC Press.

There are two general classes of IR spectrometers—dispersive and non-dispersive. In a typical dispersive IR spectrometer, radiation from a broadband source passes through the sample and is dispersed by a monochromator into component frequencies. The beams then fall on a detector, typically a thermal or photon detector, which generates an electrical signal for analysis. Fourier Transform IR spectrometers (FTIR) have replaced the dispersive IR spectrometer due to their superior speed and sensitivity. FTIR eliminates the physical separation of optical component frequencies by using a moving mirror Michelson interferometer and taking the Fourier transform of the signal.

Conversely, in the non-dispersive IR (NDIR) spectrometer, instead of sourcing a broad IR spectrum for analyzing a range of sample gases, the NDIR sources a specific wavelength, which corresponds to the absorption wavelength of the target sample. This is accomplished by utilizing a relatively broad IR source and using spectral filters to restrict the emission to the wavelength of interest. For example, NDIR is frequently used to measure carbon monoxide (CO), which absorbs IR energy at a wavelength of 4.67 microns. By carefully tuning the IR source and detector during design, a high volume production CO sensor is manufactured. This is particularly impressive, as carbon dioxide is a common interferent and has an IR absorption wavelength of 4.26 microns, which is very close to that of CO.

NDIR sensors promise low cost (less than $200), no recurring costs, good sensitivity and selectivity, no calibration and high reliability. They are small, draw little power and respond quickly (less than 1 minute). Warm up time is nominal (less than 5 minutes). Unfortunately, they only detect one target gas. To detect more gases additional spectral filters and detectors are required, as well as additional optics to direct the broadband IR source:

Ion Mobility Spectrometry (IMS)

Ion Mobility Spectrometry (IMS) separates ionized molecular samples on the basis of their transition times when subjected to an electric field in a tube. As the sample is drawn into the instrument, it is ionized by a weak radioactive source. The ionized molecules drift through the cell under the influence of an electric field. An electronic shutter grid allows periodic introduction of the ions into the drift tube where they separate based on charge, mass, and shape. Smaller ions move faster than larger ions through the drift tube and arrive at the detector sooner. The amplified current from the detector is measured as a function of time and a spectrum is generated. A microprocessor evaluates the spectrum for the target compound, and determines the concentration based on the peak height.

IMS is an extremely fast method and allows near real time analysis. It is also very sensitive, and should be able to measure all the analytes of interest. IMS is moderate in cost (several thousand dollars) and larger in size and power consumption.

Metal Oxide Semiconductor (MOS) Sensors

Metal Oxide Semiconductor (MOS) sensors utilize a semiconducting metal-oxide crystal, typically tin-oxide, as the sensing material. The metal-oxide crystal is heated to approximately 400° C., at which point the surface adsorbs oxygen. Donor electrons in the crystal transfer to the adsorbed oxygen, leaving a positive charge in the space charge region. Thus, a surface potential is formed, which increases the sensor's resistance. Exposing the sensor to deoxidizing, or reducing, gases removes the surface potential, which lowers the resistance. The end result is a sensor that changes its electrical resistance with exposure to deoxidizing gases. The change in resistance is approximately logarithmic.

MOS sensors have the advantage of being extremely low cost (less than $8 in low volume) with a fast analysis time (milliseconds to seconds). They have long operating lifetimes (greater than five years) with no reported shelf life issues.

Photo-Ionization Detectors (PID)

Photo-Ionization Detectors rely on the fact that all elements and chemicals can be ionized. The energy required to displace an electron and ‘ionize’ a gas is called its Ionization Potential (IP), measured in electron volts (eV). A PID uses an ultraviolet (UV) light source to ionize the gas. PIDs are sensitive (low ppm), low cost, fast responding, portable detectors. They also consume little power.

The energy of the UV light source used by a PID must be at least as great as the IP of the sample gas. For example, benzene has an IP of 9.24 eV, while carbon monoxide has an IP of 14.01 eV. For the PID to detect the benzene, the UV lamp must have at least 9.24 eV of energy. If the lamp has an energy of 15 eV, both the benzene and the carbon monoxide would be ionized. Once ionized, the detector measures the charge and converts the signal information into a displayed concentration. Unfortunately, the display does not differentiate between the two gases, and simply reads the total concentration of both summed together.

Three UV lamp energies are commonly available: 9.8, 10.6 and 11.7 eV. Some selectivity can be achieved by selecting the lowest energy lamp while still having enough energy to ionize the gases of interest. The largest group of compounds measured by a PID are the organics (compounds containing carbon), and they can typically be measured to parts per million (ppm) concentrations. PIDs do not measure any gases with an IP greater than 11.7 eV, such as nitrogen, oxygen, carbon dioxide and water vapor. The CRC Press Handbook of Chemistry and Physics includes a table listing the IPs for various gases.

Microcantilever Sensors

Microcantilever sensors are hairlike, silicon-based devices that are at least 1,000 times more sensitive and smaller than currently used sensors. The working principle for most microcantilever sensors is based on a measurement of displacement. Specifically, in biosensor applications, the displacement of a cantilever-probe is related to the binding of molecules on the (activated) surface of the cantilever beam, and is used to compute the strength of these bonds, as well as the presence of specific reagents in the solution under consideration (Fritz, J. et al., “Translating biomolecular recognition into nanomechanics,” Science, 288:316-318 (2000); Raiteri, R. et al., “Sensing of biological substances based on the bending of microfabricated cantilevers,” Sensors and Actuators B, 61:213-217 (1999)). It is clear that the sensitivity of these devices strongly depends on the smallest detectable motion, which poses a constraint on the practically vs. theoretically achievable performance.

Microcantilever sensors are highly advantageous in that they can detect and measure relative humidity, temperature, pressure, flow, viscosity, sound, ultraviolet and infrared radiation, chemicals, and biomolecules such as DNA, proteins, and enzymes. Microcantilever sensors are rugged, reusable, and extremely sensitive, yet they cost little and consume little power. Another advantage in using the sensors is that they work in air, vacuum, or under liquid environments.

Portable Device

According to the subject invention, a portable device is provided that includes at least one form of sensor technology described above. Preferably, the portable device is a handheld instrument for use in sensing the presence of one or more target markers in a sample (such as a sample of biological fluid or of headspace from a prescription drug container). The portable device can also include any means known to the skilled artisan useful in providing a sample to the sensor(s) of the portable device. Contemplated sample providing means include, but are not limited to, a wand, chamber, dish, plate, well, assay sheet or film, and dipstick, all which provide means in which samples can be received for analysis using the sensor(s) of the invention.

In one embodiment, the sample providing means is a chamber for collecting samples of exhaled breath. A variety of systems have been developed to collect and monitor exhaled breath components, particularly gases. For example, U.S. Pat. No. 6,010,459 to Silkoff describes a method and apparatus for the measurement of components of exhaled breath in humans. Various other apparatus for collecting and analyzing expired breath include the breath sampler of Glaser et al, U.S. Pat. No. 5,081,871; the apparatus of Kenny et al, U.S. Pat. No. 5,042,501; the apparatus for measuring expired breath of infants of Osborn, U.S. Pat. No. 4,202,352; the blood alcohol concentration measuring from respiratory air method of Ekstrom, U.S. Pat. No. 5,971,937, and the instrument for parallel analysis of metabolites in human urine and expired air of Mitsui et al., U.S. Pat. No. 4,734,777. Pulmonary diagnostic systems including computerized data analysis components also are known, see Snow et al., U.S Pat. No. 4,796,639.

Signals obtained from sensor technology within the portable device are transmitted to a processing means located within the portable device for signal processing. The processing means can also be responsible for maintenance of acquired data as well as the maintenance of the entire portable device itself. The processing means can also detect and act upon user input via user interface means known to the skilled artisan (such as a keyboard, or an interactive graphical monitor). In a related embodiment, the portable device can include a display (such as a liquid crystal display, a monitor, etc.) for communicating the portable device's operating modes and/or results of the portable device's sensing.

According to the subject invention, the processing means can be implemented as an application specific integrated circuit (ASIC), a digital signal processor (DSP), a controller, a microprocessor, or other circuits designed to perform the functions described herein.

In certain embodiments, the processing means can also include one or more memory devices to store program codes, data, and other configuration information. Suitable memory devices include a random-access memory (RAM), a dynamic RAM (DRAM), a FLASH memory, a read only memory (ROM), a programmable read only memory (PROM), an electrically programmable ROM (EPROM), an electrically erasable and programmable PROM (EEPROM), and other memory technologies. The size of the memory device(s) is application dependent, and can be readily expanded as needed.

In one embodiment, the processing means executes program codes that coordinate various operations of the portable device. The program codes include interaction software that assists the user in selecting the operating modes and methods and to initiate the analysis of a sample using the sensor(s) of the portable device. The program codes can also include software that performs analysis functions for information provided by the sensor(s) regarding a sample as well as software that enables prescribed event analysis. For example, a calendar program code can be provided that allows the processing means to store and retrieve scheduling information (such as from the memory device(s) regarding when a prescribed event occurred).

In controlling various aspects of the portable device, the processing means can control such effects as temperature, humidity, pH, salinity, etc. of the sensor(s) technology and/or sample. For example, each sensor array and sample chamber can include a suitable thermoelectric device for use in heating or cooling.

After the portable device of the invention performs a test or operation, the user (,patient, pharmacist, physician) is optionally presented with concise results.

In another embodiment, the device further includes a data filter, a built-in algorithm, and an event indicator. The data filter, built-in algorithm, and event indicator enable the portable device to perform complex functions and capabilities. For example, the data filter can be provided to parse through the data provided by the sensor(s) to determine whether an event has occurred as prescribed. An event indicator can be connected to the data filter that is responsive to detection of the event by the data filter (such as, where the event is patient administration of a medication at a specified time as prescribed). In certain related embodiments, the event indicator can include an event indicator monitor which monitors the event indicator to determine. whether the user has performed a prescribed event (such as the number of times a patient has taken a medication at a specified time as prescribed). In other embodiments in which simplified electronics is provided, complex. functions and capabilities of the portable device are optionally set up and driven from a host computer using PC based software.

In another embodiment, where the sensor technology comprises known e-nose technology, the processing means can correlate collected data with data representing a set of previously collected standards stored in a memory device (for example, RAM). This comparison facilitates identification of target marker(s) present in the sample providing means (such as a chamber, wand, plate, etc.) and determination of the quantity or concentration of such target markers, as well as detection of temporal changes in such identities and quantities. Various analyses suitable for identifying target marker(s) and quantifying concentration include principal component analysis, Fischer linear analysis, artificial neural networks (ANNs), genetic algorithms, fuzzy logic, pattern recognition, and other algorithms. After analysis is completed, the resulting information can be displayed on a display and/or transmitted to a central computer of the invention via electronic communication.

Alternatively, analysis can be performed by the central computer of the invention. For example, sensor(s) information regarding a sample can be stored by the processing means of the portable device and upon transmittal to a central computer (for example, at a pharmacy or physician's office), the central computer will analyze the sensor(s) information using its own processing means. The processing means of either the central computer or the portable device can be a processor, a DSP processor, a specifically designed ASIC, or other circuits designed to perform the analysis functions for identifying target markers present in a sample, determining the quantity or quality of target markers, and detecting temporal changes in such identity or quantity of target marker in a sample. In certain embodiments, the processing means can be a general-purpose processor that executes program codes written to perform the required analysis function.

The processing means ofthe: central computer and/or portable device of the invention can further direct data acquisition, perform digital signal processing, and/or provide control over serial peripheral devices (via serial peripheral interface), input/output devices (I/Os), serial communications (via serial communication interface), and other peripheral devices. Serial peripheral devices that can be controlled by the processing means include, but are not limited to, an analog-to-digital converter and digital-to-analog converter, a 32K external EPROM (with the capability to expand to 64K), a 32K RAM with integrated real time clock and battery back up, a 2×8-character dot matrix display, and others. I/Os that can be controlled include temperature probes, humidity probes, light emitting diodes, and others.

The processing means of the central computer and/or portable device of the invention can further control peripheral devices such as the display and sensor technology (such as the valve assembly and the pump used in a SAW sensor). The processing means can also monitor input devices (such as push button switches on a keyboard) and further provides digital communication via an electronic communication device (for example via a modem, Ethernet card, wireless communication devices, etc.), which enables either direct or remote communication between the portable device and the central computer.

In a method of use, a known reference sample is provided to the sample providing means (such as a chamber, wand, plate) of the portable device. The known sample is provided to enable to processing means to identity a reference sample. In one embodiment, a known reference sample is provided in a cartridge, wherein the cartridge can be replaced periodically.

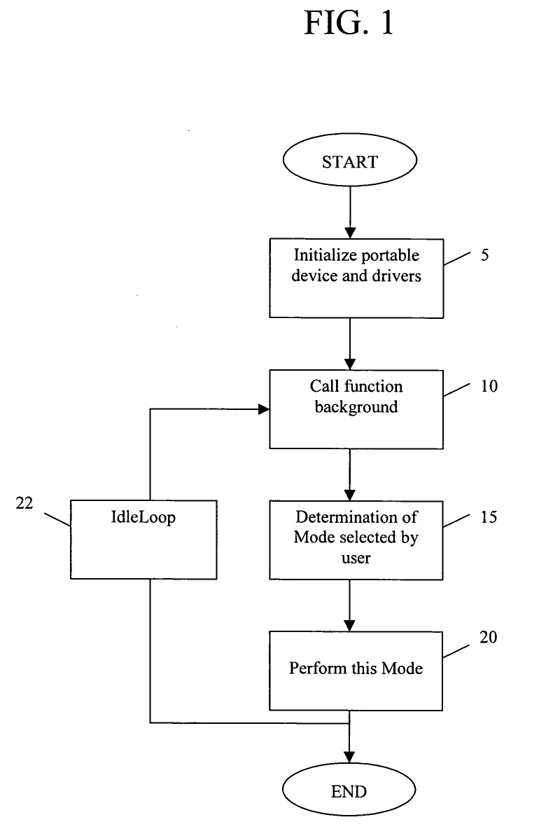

FIGS. 1-4 depict an embodiment of suitable flowcharts of the functional steps performed by the subject invention's portable device and central computer in implementing the measurement and analysis procedures outlined generally above. These flowcharts show how the portable device is initialized and then controlled through its various operating modes. In one embodiment for monitoring a patient's compliance with a prescribed regimen, these operating modes include: 1) a function background mode, in which the device is calibrated by exposing it to samples of marker(s) of known identity, 2) a Target mode, in which the device is exposed to a samples of unknown identity, and 3) a Purge mode, in which the device is purged of resident samples. In another embodiment, in which a prescription drug's origin is to be verified, the operating modes. can include: 1) a background function mode, in which the portable device is calibrated by exposing it to samples of marker(s) of known identity, 2) a Target mode, in which the device is exposed to samples of unknown identity, and 3) a Purge mode, in which the device is purged of resident samples.

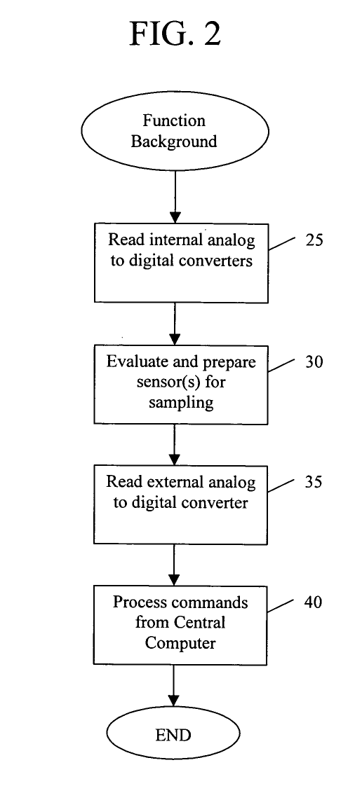

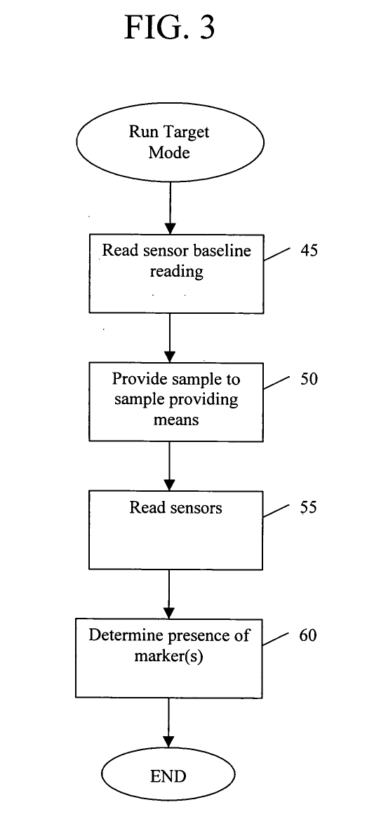

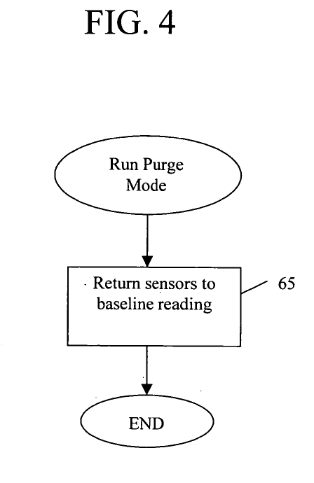

FIG. 1 shows a flow diagram of an embodiment of the main program menu of the portable device. Initially, the portable device's various electronic elements (such as the display and various internal data registers) are initialized or reset at step 5. A function background subroutine is then executed at step 10. This subroutine is further described in FIG. 2. After executing the function background subroutine, the program proceeds to a step 15 in which the processor determines which operating mode (Mode) is being selected by the user. Thereafter, the program proceeds to implement the selected Mode in step 20, which are depicted in the following FIGS. 3-4. When an operating mode has not yet been selected, the program returns via an idle loop 22 to step 10 and re-executes the function background subroutine.

FIG. 2 shows a flow diagram of an embodiment of the function background subroutine (step 10). At step 25, signals indicative of the measurements and parameters selected by the user (for example, the temperature and humidity within the sample providing means) are read from the analog-to-digital converters (ADCs) that are configured to detect the input devices (also referred to as the internal ADCs). The step of 30 evaluates the status of the sensors and prepares them for sampling based on the signals from the internal ADCs. Signals indicative of sensor status/preparedness are read from the instrumentation ADCs (also referred to as the external ADCs) at step 35. Finally, at step 40, the processing means processes any commands received from the central computer via known communication (for example, via digital communication) methods. Such commands can include, for example, programming information about the identity of target markers to be detected by the sensor(s) of the portable device during the target operating mode. Alternatively, the of 40 can be one in which the portable device is calibrated by exposing it to samples of marker(s) of known identity and providing readings from step 35 to the processing means. The function background subroutine then terminates.

FIG. 3 shows a flow diagram of a target mode subroutine of the invention. At step 45, the most recently updated set of measurements from the external ADCs is retrieved. These measurements represent the status/preparedness of the sensor(s) of the portable device. Next, a sample (such as a sample of bodily fluid, headspace from a medication container, medication container filler, etc.) is provided to the sample providing means of the portable device at step 50. A new set of measurements is then retrieved from the external ADCs at step 55. This new set of measurements indicates the output from the sensor(s) as they respond to the sample that has been provided. In step 60, sensor output is analyzed to determine target marker detection in the sample of a target marker. In certain embodiments, after determining the presence of the target marker, the information is saved and/or communicated to a central computer for use in ensuring compliance with a prescription regimen. The target operation mode then terminates and returns to the idle loop (step 22 of FIG. 1).

FIG. 4 shows a flow diagram of an embodiment of the purge mode subroutine. At step 65, the sensor(s) of the portable device are conditioned to return to the baseline reading. Alternatively, the processing means may also be programmed to remove all information regarding the target marker from permanent memory. The program then returns to the idle loop (step 22 of FIG. 1).

In certain aspects of the invention, the portable device is designed using modular sections. For example, the sensor(s), processing means, memory device(s), and/or others can optionally be disposed within a module that can be installed or swapped, as necessary. The modular design provides many advantages, some of which are related to the following characteristics: exchangeable, removable, replaceable, upgradable, and non-static. The modular design can also provide for disposable modules.

In certain embodiments, the modular design can also provide improved performance. The various modules (for example, the sensor(s) or sample providing means) can be designed to provide accurate measurement of a particular set of test samples. Different modules can be used to measure different samples. Thus, performance is not sacrificed by the use of a portable device. For example, to sense high molecular weight marker(s), a certain particular sensor(s) technology is plugged in (for example an e-nose chip). Then, to analyze lower molecular weight marker(s), another sensor(s) technology may be plugged in (such as SAW technology).

The modular design can also result in a cost effective portable device design. Since some of the components can be easily replaced, it is not necessary to dispose the entire portable device if a particular component wears out. Only the failed components are to be replaced.

In certain embodiments, the modular design can also provide an upgradable design. For example, the processing means or memory device (individually or in combination) can be disposed within an electronic modular unit that can be upgraded with new technologies, or as required by the particular application. Additional memory can be provided to store more data, by simply swapping out memory modules. Also, analysis algorithms can be included in a program module that inserts into the portable device. According to the subject invention, program modules can then be swapped as desired.

The portable device, according to the subject invention, can include any known identification systems such as, but not limited to, the use of a “biometric” identification system, an electronic coding system (such as a password protected system), a lock-and-key identification system, etc. With the lock-and-key identification system, the portable device comprises a lock that prevents the user (such as the patient, pharmacist, etc.) access/use of the portable device unless an appropriate compatible key is provided. The user is provided with a key prior to ensure secure use of the portable device.

With the electronic coding system such as a password protection method, the user must enter a specific password (for example, through a keyboard attached to the portable device) to initiate use of the portable device. The password is then transmitted to the processing means (of the portable device) and/or the analyzing means (of the central computer), where it is compared to a password database that contains a password for all users that have been registered by a system administrator to access the portable device. If a match is found, the processing means and/or analyzing means permits the user onto the portable device and the user can use the portable device as designated for that user.

As used throughout this disclosure, the term “biometric” generally refers to any bodily parameter unique to each user. Examples of biometrics include fingerprints, hand geometry, facial geometry, retinal scan, voice, body odor or any other characteristic that distinguishes one person from another. Biometrics can be detected, measured, and/or scanned by known devices such as those provided by Identicator Technologies, Corp., which has introduced a fingerprint sensor device that connects to a computer system. A user places his or her finger on the surface of the device and an image is captured of the user's fingerprint. That fingerprint image is provided to the computer system. The computer processes the fingerprint image and generates a “template” of the image, which is a value representative of the raw image.

With biometric identification systems, a user of the subject invention is first enrolled as a registered user and an image is captured of the user's biometric feature (such as a fingerprint, retina, voice, etc.) and a template is generated therefrom. A password is then assigned to the user. The password and template are stored in a database (in the processing means of the portable device and/or analyzing means of the central computer) and indexed by user name. The database thus contains passwords and biometric templates for all users wishing to log on using the biometric identification mechanism. During the log on process, the processing means and/or analyzing means compares the template generated to templates previously stored in the database. If a match is found, the processing means/analyzing means selects the password that is stored with the matching biometric template and uses the password to provide user access to use of the portable device.

Central Computer

The central computer, according to the subject invention, is housed within a facility that is remotely located from the patient to be monitored. In a preferred embodiment, the central computer is housed within a pharmacy facility while a patient is located at home.

According to the subject invention, information is provided to the central computer and/or portable device regarding the prescribed medication and/or medication origin prior to sampling by the portable device. Information that can be provided to the central computer, as well as the portable device, includes but is not limited to the following: information regarding the prescribed regimen for the medication; information regarding the markers of the medication that are detectable in a patient's bodily fluid; information regarding markers indicative of a medication's origin; information regarding medication side effects.

In one embodiment, the central computer comprises a means for storing and means for outputting processed data. The central computer includes any digital instrumentation capable of processing signals from the portable device of the invention (such as SAW sensor generated signals). Such digital instrumentation, as understood by the skilled artisan, can process communicated signals by applying algorithm and filter operations of the subject invention. Alternatively, the central computer can process data that has already been analyzed and communicated from the portable device. Preferably, the digital instrumentation is a microprocessor, a personal desktop computer, and/or a laptop. The central computer can be a general purpose or application specific computer.

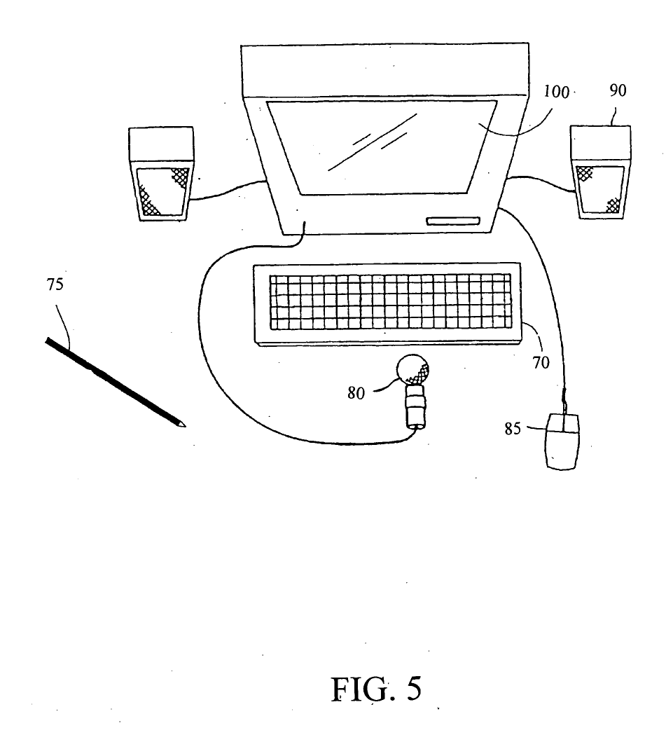

Referring to FIG. 5, the central computer of the subject invention can contain at least one user-interface device including, but not limited to, an input device 70, stylus 75, microphone 80, mouse 85, speaker 90, and monitor 100. Additional user-interface devices contemplated herein include touch screens, strip recorders, joysticks, printers, and rollerballs.

Preferably, the central computer comprises a central processing unit (CPU) having sufficient processing power.to perform program codes and algorithm operations in accordance with the subject invention. The program codes and algorithm operations, including the filtering, analysis, and monitoring operations, can be embodied in the form of computer processor usable media, such as floppy diskettes, CD-ROMS, zip drives, non-volatile memory, or any other computer-readable storage medium, wherein the computer program code is loaded into and executed by the central computer. Optionally, the program codes and/or operational algorithms of the subject invention can be programmed directly onto the CPU using any appropriate programming language, preferably using the C programming language.

The central computer can also include a neural network for pattern recognition. Artificial Neural Networks ANNs are self learning; the more data presented, the more discriminating the instrument becomes. By running many standard samples and storing results in computer memory, the application of ANN enables the device to “understand” the significance of the sensor array outputs better and to use this information for future analysis (for example, to analyze whether the prescribed medication is being metabolized properly over a period of time). “Learning” is achieved by varying the emphasis, or weight, that is placed on the output of one sensor versus another. The learning process is based on the mathematical, or “Euclidean,” distance between data sets. Large Euclidean distances represent significant differences in. sample-to-sample aroma characteristics.

In certain embodiments, the central computer comprises a memory capacity sufficiently large to perform program codes and/or algorithm operations in accordance with the subject invention. The memory capacity of the invention can support loading a computer program code via a computer-readable storage media, wherein the program contains the source code to perform the program codes and/or operational algorithms of the subject invention. Optionally, the memory capacity can support directly programming the CPU to perform the operational algorithms of the subject invention. A standard bus configuration can transmit data between the CPU, memory, ports and any communication devices.

In addition, as understood by the skilled artisan, the memory capacity of the central computer can be expanded with additional hardware and with saving data directly onto external mediums including, for example, without limitation, floppy diskettes, zip drives, non-volatile memory and CD-ROMs.

The central computer can further include the necessary hardware and software to provide analyzed sensor(s) information into an output form readily accessible by the pharmacist, trained physician, nurse practitioner, midwife, or technician. For example, without limitation, an audio device in conjunction with audio speakers can relay sample analysis results into an audio signal, and/or a graphical interface can display results in a graphical form on a monitor and/or printer. Further, the central computer can also include the necessary software and hardware to receive, route and transfer data to and from a remote location in which the portable device is in use.

The subject invention can be practiced in a variety of situations. The central computer means can directly or remotely connect to a portable device. In one embodiment, the subject invention is practiced directly in a pharmacy. In another embodiment, the subject invention is practiced in a remote setting, for example, personal residences, mobile clinics, vessels at sea, rural villages and towns.

To ensure patient privacy, security measures, such as encryption software and firewalls, can be employed in the central computer. Optionally, clinical data can be transmitted as unprocessed or “raw” signal(s) and/or as processed signal(s). Advantageously, transmitting raw signals allows any software upgrades to occur at the location where the central computer is located. In addition, both historical clinical data and real-time clinical data can be transmitted.

Communication devices such as wireless interfaces, cable modems, satellite links, microwave relays, and traditional telephonic modems can transfer data and/or analyzed data from a portable device to a central computer via a electronic communication (such as a network). Networks available for transmission of data include, but are not limited to, local area networks, intranets and the open Internet. A browser interface, for example, NETSCAPE NAVIGATOR or INTERNET EXPLORER, can be incorporated into communications software to view the transmitted data.

In one embodiment, two-way communication between the portable device and the central computer is permitted. Two-way communication may permit the central computer to upload a set of questions or messages for presentation to a patient via the portable device. For example, in the case where the portable device is used to monitor the patient's compliance with a prescribed regimen, a missed sampling of bodily fluid might cause a pharmacist to send a customized question for presentation to the patient: “Have you forgotten to take your medication today?” Alternatively, where a patient has questions regarding a prescription regimen, a patient may send a question for presentation to the pharmacist: “If I take this medication, will it affect my blood pressure medication?” Such customized questions could be presented the next time the portable device/central computer is accessed or can be presented to the patient/pharmacist in real time. Additionally, a customized message may be scheduled for delivery at certain times (for example, half an hour after prescribed times in which a sample is to be taken and analyzed). Further, the messages may be selected from a list.

The central computer of the subject invention can function in a real-time setting to continuously communicate with a portable device so as to provide accurate data to the user regarding patient compliance with a prescribed regimen. Alternatively, the central computer of the subject invention can function on a schedule to basis, where communication between the portable device and central computer is regimented. Or, the central computer of the subject invention can communicate with a portable device pursuant to manual initiation by the user (such as a pharmacist, patient, technician, etc.). For example, where a pharmacist would like to assess the origin of a drug, a portable device can be applied to the headspace of the container for the drug and the pharmacist can then initiate communication between the portable device and the central computer to verify the origin of the drug.

Target Markers

In accordance with the present invention, markers (or taggants) useful as an indication of prescribed drug presence in a patient and/or of prescription drug origin include the following olfactory markers, without limitation: dimethyl sulfoxide (DMSO), acetaldehyde, acetophenone, trans-Anethole (1-methoxy-4-propenyl benzene) (anise), benzaldehyde (benzoic aldehyde), benzyl alcohol, benzyl cinnamate, cadinene, camphene, camphor, cinnamaldehyde (3-phenylpropenal), garlic, citronellal, cresol, cyclohexane, eucalyptol, eugenol, eugenyl methyl ether, butyl isobutyrate (n-butyl 2, methyl propanoate) (pineapple), citral (2-trans-3,7-dimethyl-2,6-actadiene-1-al), menthol (1-methyl-4-isopropylcyclohexane-3-ol), and α-Pinene (2,6,6-trimethylbicyclo-(3,1,1)-2-heptene). These markers are preferred since they are used in the food industry as flavor ingredients and are permitted by the Food and Drug Administration As indicated above, olfactory markers for use in the present invention can be selected from a vast number of available compounds (see Fenaroli's Handbook of Flavor Ingredients, 4^{th} edition, CRC Press, 2001) and use of such other applicable markers is contemplated herein.

The markers of the invention also include additives that have been federally approved and categorized as GRAS (“generally recognized as safe”), which are available on a database maintained by the U.S. Food and Drug Administration Center for Food Safety and Applied Nutrition. Markers categorized as GRAS that are readily detectable in exhaled breath include, but are not limited to, sodium bisulfate, dioctyl sodium sulfosuccinate, polyglycerol polyricinoleic acid, calcium casein peptone-calcium phosphate, botanicals (for example, chrysanthemum; licorice; jellywort, honeysuckle; lophatherum, mulberry leaf; frangipani; selflheal; sophora flower bud, etc.), ferrous bisglycinate chelate, seaweed-derived calcium, DHASCO (docosahexaenoic acid-rich single-cell oil) and ARASCO (arachidonic acid-rich single-cell oil), fructooligosaccharide, trehalose, gamma cyclodextrin, phytosterol esters, gum arabic, potassium bisulfate, stearyl alcohol, erythritol, D-tagatose, and mycoprotein.

It is known that certain GRAS molecules have the ability to be absorbed and excreted (such as in exhaled breath). For example, certain GRAS molecules can be absorbed by the patient via a mucus membrane (for example, gastrointestinal mucosa) and then excreted in exhaled breath. Further, certain GRAS compounds are available wherein metabolism is: required (such as by the cytochrome p450 enzyme system) to generate a marker that can be detected in exhaled breath. Such GRAS molecules will be useful in circumventing patient attempts to fake taking a medication as prescribed. The following Table 1 provides a list of GRAS compounds that may be used in accordance with the subject invention.

TABLE 1
List of Markers based on GRAS

| DOCTYPE | DOCNUM | MAINTERM | CODE* | REGNUM |
|---|---|---|---|---|
| ASP | 311 | Dibenzyl Ether | 000103- | 172.515 |
| | | | 50-4 | |
| ASP | 328 | Difurfuryl Ether | 004437- | |
| | | | 22-3 | |
| ASP | 445 | Ethylene Glycol | 000111- | 178.1010 |
| | | Monobutyl Ether | 76-2 | 175.105 |
| | | | | 176.210 |
| | | | | 177.1650 |
| ASP | 557 | Furfuryl Methyl Ether | 013679- | |
| | | | 46-4 | |
| ASP | 775 | Isoeugenyl Benzyl | 000120- | 172.515 |
| | | Ether | 11-6 | |
| ASP | 776 | Isoeugenyl Ethyl Ether | 007784- | 172.515 |
| | | | 67-0 | |
| ASP | 778 | Isoeugenyl Methyl | 000093- | 172.515 |
| | | Ether | 16-3 | |
| ASP | 1023 | Methyl Phenethyl | 003558- | |
| | | Ether | 60-9 | |
| ASP | 1097 | Beta-Naphthyl | 002173- | |
| | | Isobutyl Ether | 57-1 | |
| ASP | 3017 | Vanillyl Butyl Ether | 082654- | |
| | | | 98-6 | |
| NIL | 2028 | Dimethylethanolamine | 000108- | 173.20 |
| | | | 01-0 | 175.105 |
| | | | | 175.300 |
| EAF | 3383 | Isopentyl- | 035448- | |
| | | ideneisopentylamine | 31-8 | |

*CAS, RN, OR OTHER CODE

The definitions of the labels that are provided in Table 1 are as follows:

| Label | Definition | |
|---|---|---|
| DOCTYPE | An indicator of the status of the toxicology information available for | |
| | the substance in PAFA (administrative and chemical information is | |
| | available on all substances) | |
| | ASP | Fully up-to-date toxicology information has been sought |
| | EAF | There is reported use of the substance, but it has not yet |
| | | been assigned for toxicology literature research |
| | NEW | There is reported use of the substance, and an initial |
| | | toxicology literature search is in progress |
| | NIL | Although listed as added to food, there is no current |
| | | reported use of the substance and, therefore, although |
| | | toxicology information may be available in PAFA, it is not |
| | | being updated |
| | NUL | There is no reported use of the substance and there is no |
| | | toxicology information available in PAFA |
| | BAN | The substance was formerly approved as a food additive but |
| | | is now banned; there may be some toxicology data available |

| | |
|---|---|
| DOCNUM | PAFA database number of the Food Additive Safety Profile volume |
| | containing the printed source information concerning the substance |
| MAINTERM | The name of the substance as recognized by CFSAN |
| CAS, RN, OR | Chemical Abstract Service (CAS) Registry Number for the substance |
| OTHER CODE | or a numerical code assigned by CFSAN to those substances that do |
| | not have a CAS Registry Number (888nnnnnn or 977nnnnn-series) |
| REGNUM | Regulation numbers in Title 21 of the U.S. Code of Federal |
| | Regulations where the chemical appears |

As described above, markers of the invention are detected by their physical and/or chemical properties, which does not preclude using the prescribed therapeutic drug itself as its own marker. Where the therapeutic drug is the marker itself, the drug can be manufactured to include products and compounds that enhance detection of the marker(s) using sensors of the invention. In certain instances, markers (that are the therapeutic drug itself) that are poorly soluble in water demonstrate enhanced volatility and facilitate detection in the breath.

According to the present invention, prescribed medications can be administered to a patient via a variety of routes including, for example, orally-administrable forms such as tablets, capsules or the like, or via parenteral, intravenous, intramuscular, transdermal, buccal, subcutaneous, suppository, or other route.

According to the subject invention, after taking a prescribed drug (wherein the therapeutic drug is the marker or the therapeutic drug is manufactured to include a detectable marker), a patient provides a sample of bodily fluid to a portable device of the invention. Marker detection can occur under several circumstances in the portable device. In one example, where the drug is administered orally, the marker can “coat” or persist in the mouth, esophagus and/or stomach upon ingestion and be detected with exhalation (similar to the taste or flavor that remains in the mouth after eating a breath mint).

In one embodiment, where the prescribed drug is administered orally, the drug may react in the mouth or stomach with acid or enzymes to produce or liberate the marker(s) that can then be detected upon exhalation. Thirdly, the drug and/or marker can be absorbed in the gastrointestinal tract and be excreted in the lungs (for example, alcohol is rapidly absorbed and detected with a Breathalyzer).

In another embodiment, the marker(s) of the invention is concurrently administered with a therapeutic drug (for example, the marker is provided in a pharmaceutically acceptable carrier, where the marker is in the medication coating composed of rapidly dissolving glucose and/or sucrose). In a related embodiment, markers are provided for commonly diverted prescription drugs including, but not limited to, narcotic analgesics (such as Darvon, Demerol, Dilaudid, Fentanyl, Methadose, MSIR, Nubain, Oxycontin, Roxanol, Stadol); narcotic analgesics in combination with other medications (such as Vicodin; Lorcet; Tylox; Percocet); medications to treat various mental conditions or disorders (such as Diazepam, Paroxetine, Sertraline, Fluoxetine); medications for treating erectile dysfunction (such as Viagra), medications for treating weight problems (such as Meridia); cerebral nervous system depressants (such as Mebaral, Nembutal, Librium, Xanax, Halcion, ProSom); medications to treat diarrhea (such as Lomotil); and stimulants (such as Adderal; Dexedrine, Ritalin; Focalin; Provigil). Although such pharmaceutical drugs have legitimate medical purposes, they are often illegally diverted for recreational use, which costs the federal government and states billions of dollars in areas such as law enforcement, health care, social services, and court costs.

In a preferred embodiment, the therapeutic drug is provided in the form of a pill, whose coating includes at least one marker in air-flocculated sugar crystals. This would stimulate salivation and serve to spread the marker around the oral cavity, enhancing the lifetime in the cavity. Since the throat and esophagus could also be coated with the marker as the medication is ingested, detection of the marker is further enhanced.

Thus, when a drug is administered to a patient, the preferred embodiment of the invention detects and quantifies a therapeutic drug marker almost immediately in the exhaled breath of the patient (or possibly by requesting the patient to deliberately produce a burp) using a sensor (for example, an electronic nose). Certain drug compositions might not be detectable in the exhaled breath. Others might have a coating to prevent the medication from dissolving in the stomach. In both instances, as an alternate embodiment, a non-toxic olfactory marker (such as volatile organic vapors) can be added to the pharmaceutically acceptable carrier (for example, the coating of a pill, in a separate fast dissolving compartment in the pill, or solution, if the drug is administered in liquid or suspension form) to provide a means for identifying/quantifying the marker in exhaled breath and thus determine whether a patient has taken a medication in accordance with a prescribed regimen.

Preferably the marker will coat the oral cavity or esophagus or stomach for a short while and be exhaled in the breath (or in a burp). For drugs administered in the form of pills, capsules, and fast-dissolving tablets, the markers can be applied as coatings or physically combined or added to therapeutic drug. Markers can also be included with therapeutic drugs that are administered in liquid form (such as via syrups, via inhalers, or other dosing means).

In other embodiments, prescribed medications are administered intravenously. With intravenous administration, a prescribed medication is provided directly into a patient's bloodstream. An intravenously administered drug may bind to proteins circulating in the blood, be absorbed into fat or exist in a “free” form. The sensor(s) of the invention detect the “free” form of the drug in embodiments in which an intravenously administered medication itself is the detectable marker use. Free drug or a metabolite of the drug can be excreted in the urine or the digestive tract or in exhaled breath. Alternatively, sensor(s) of the invention can detect any marker that is added to an intravenous prescribed medication (for example, the medication can be manufactured to include a GRAS marker). Such markers can be released into any bodily fluid for detection by the sensor(s) of the invention.

In further embodiments where the prescribed medication is provided via parenteral, intramuscular, transdermal, buccal, subcutaneous, or suppository routes, the marker can be the medication itself or a detectable compound that is added to the medication to ensure detection by the portable device.

In another embodiment, markers that can be detected by the portable device include those that are indicative of controlled substances (such as controlled substances that are either prescribed or not prescribed). For example, in addition to marker(s) of prescribed medications, the sensor(s) of the invention can detect markers representative of, but not limited to, illicit, illegal, and/or controlled substances including drugs of abuse (such as amphetamines, analgesics, barbiturates, club drugs, cocaine, crack cocaine, depressants, designer drugs, ecstasy, Gamma Hydroxy Butyrate—GHB, hallucinogens, heroin/morphine, inhalants, ketamine, lysergic acid diethylamide—LSD, marijuana, methamphetamines, opiates/narcotics, phencyclidine—PCP, psychedelics, Rohypnol, steroids, and stimulants).

The markers of the invention could be used for indicating specific drugs or for a class of drugs. For example, a patient may be taking an anti-depressant (tricyclics such as nortriptyline), antibiotic, an antihypertensive agent (for example, clonidine), pain medication, and an anti-reflux drug. One marker could be used for antibiotics as a class, or for subclasses of antibiotics, such as erythromycins. Another marker could be used for antihypertensives as a class, or for specific subclasses of antihypertensives, such as calcium channel blockers. The same would be true for the anti-reflux drug. Furthermore, combinations of marker substances could be used allowing a rather small number of markers to specifically identify a large number of medications.

A system of the invention comprises a central computer and a portable device, wherein the portable device includes at least one sensor and one sample providing means. The sensors of the present invention are in communication with the sample providing means to appropriately monitor the presence of target marker(s). For example, where exhaled breath or headspace from a prescription medication container is to be examined by the portable device, the sensors are in flow communication with the appropriate tubes, valves, etc. of a breathing circuit (where the sample is exhaled breath) or sample wand (where the sample is headspace).

In one method of use, a medication is provided to a patient with a prescribed regimen and portable device of the invention. At a specified time interval after each administered dosage of medication, the patient will provide a sample of bodily fluid to the portable device. According to the subject invention, the sample of bodily fluid will be provided to the sensor(s) of the portable device. Depending on the system, information provided by the sensor(s) will be: provided directly to a central computer for analysis; analyzed by a processing means within the portable device; or stored by the processing means within the portable device for future analysis to be performed by the central computer. Detection of a target marker provides notice to the user of the medication's presence in the patient and consequently, allows assessment of whether a drug has been taken as prescribed.

In another method of use, a medication will be manufactured with a particular volatile marker (or “taggant”) for use in detecting counterfeit medications. Information regarding the taggant for a given medication is entered into the central computer of the invention. In certain embodiments, the information is provided to the user (such as the pharmacist) who then enters the taggant information into the central computer. In other embodiments, the information regarding the taggant for a given medication may be entered directly into the central computer (for example, automatically downloaded from the prescription drug distributor or manufacturer; via code information that is scanned into the computer).

At the time a pharmacist initially opens a medication container, the headspace of the container would be sampled with a portable device of the invention to detect the taggant or marker of the medication that was released into the headspace. If the sensor(s) of the portable device does not detect the proper taggant at the appropriate concentrations, the pharmacist would know that the medication is counterfeit, isolate and prevent medication distribution, and notify the proper authorities of the alleged counterfeit drug.

In a related embodiment, medication containers can be manufactured that contain a marker for use in identifying whether the drug within is the original drug produced by the manufacturer. For example, the medication containers manufactured to have a marker that is detectable by the portable device of the invention include, but are not limited to, those that have a specific volatile marker that were introduced into the interior of a medication container prior to, during, or after addition of a prescribed medication; those that were manufactured to include a specific marker on a container component (such as a bottle, a cap, etc.); and those that have packaging items (for example, cotton fillers, desiccants, etc.) that include a detectable marker.

In a method of use, the pharmacist will be provided with information regarding the marker that should be present either within the headspace of the medication container, on a packaging item, or on a container component. Information regarding the marker can. be provided to the pharmacist (for example, from the manufacturer or medication dispenser) using known communication methods including, for example, on a coded invoice; a scannable bar-code on the medication container; facsimile; voice message; electronic message to the central computer; or postal message. When the pharmacist opens the medication container, either the container headspace, packaging item, or container component will. be sampled using the portable device of the invention (depending on the information provided to the pharmacist. If the sensor(s) of the portable device does not detect the proper marker (and in certain instances, the proper marker concentration), the pharmacist would know that the medication is counterfeit, prevent medication distribution, and notify the proper authorities of the counterfeit drug.

Following are examples, which illustrate procedures for practicing the invention. These examples should not be construed as limiting. All percentages are by weight and all solvent mixture proportions are by volume unless otherwise noted.

### EXAMPLE I

Drug Diversion Prevention System

The manufacturer of a medication that has abuse and addiction potential, and thus diversion potential, adds a small amount of a GRAS compound to the matrix containing the medication at the time the drug is manufactured. The GRAS compound is chosen on the basis that it is metabolized in the liver at the time the medication is taken and because the metabolite is volatile, thus, it appears in the breath shortly after the medication is taken and absorbed in the GI tract.

At the time the medication prescription is filled by the pharmacist, a small portable (such as a handheld) device with a sensor is programmed to detect the GRAS metabolite and is given to the patient. This portable device is properly programmed to detect the GRAS compound. In one embodiment, the fingerprint of the GRAS compound to be detected is programmed into the portable device using a central computer in the pharmacy. The drug manufacturer provides the fingerprint of the GRAS compound to the central computer of the pharmacy over secure communication links or other secure means.

In one embodiment, the GRAS compound can be changed with different lots of medication so that it would be difficult for individuals trying to divert the medication. Updates of the fingerprints of the GRAS compound contained in the medication can be uploaded over a secure network to the central computer in the pharmacy from the manufacturer or a central clearinghouse run by the pharmacy.

The portable device has a fingerprint or other biometric recognition system that must be activated each time the medication is taken to verify that the patient taking the medication is the individual for which the medication was prescribed.

The portable device can also be programmed with other prescription information; such as the time the medication should be taken and can have an alert system to remind the patient when it is time to take the medication. The device can also have a system to alert health care personnel if the GRAS compound is not detected within an appropriate time period after the alert is sounded, or can merely store the number of doses taken, the determination of how the portable device would respond would be as prescribed by a physician.

Each time the patient takes a dose of medication, s/he would blow into the portable device, which includes at least one sensor that can detect the presence of the GRAS compound in exhaled breath. The portable device can include a processing means for timestamping the event (when the patient exhaled into the portable device). In certain instances, a baseline breath sample may be required prior to taking the medication.

When the patient returns to the pharmacy to refill the medication, the portable device is placed into a dock and the stored information is downloaded to the central computer. The number of doses taken by the patient should match the number of doses previously prescribed. If there is a discrepancy, the prescribing physician, or in certain instances, such as repeated discrepancies in the number of doses taken versus the number prescribed, law enforcement can be notified and additional refills withheld.

### EXAMPLE 2

Counterfeit Drug Detection System

At the time a drug known to be frequently counterfeited (usually new medications, that are expensive) is manufactured, a small amount of a marker (also referred to herein as the “taggant”), usually a GRAS compound, is added to the medication formulation. The taggant can be rotated with different lots of the medication and the fingerprint of the taggant can be uploaded to secure central computers in pharmacies throughout the U.S.

In addition to tagging medications, the medication containers that are shipped to pharmacies can contain a bar code on the label, which is identified by a scanner of the central computer to match up with the fingerprint known to be associated with a particular lot of medication. The pharmacies are provided with portable devices that can identify the taggant at the time the bottle of medication is opened. The fingerprint of the taggant is determined by the barcode on the bottle. If the fingerprint detected does not match the appropriate fingerprint stored in the central computer, the medication is deemed counterfeit.

Alternatives to adding taggant to each dose of medication could include adding a taggant inside the screw top of the bottle or adding a packet similar to a desiccant packet to the bottle of medication. In this case a tamper evident seal would have to be incorporated into the cap of the container.

It should be understood that the examples and embodiments described herein are for illustrative purposes only and that various modifications or changes in light thereof will be suggested to person skilled in the art and are to be included within the spirit and purview of this application.

We claim:

1. A system for monitoring patient compliance in taking a medication in accordance with a prescription regimen, tracking dispensed medication, and determining the origin of the prescribed medication, comprising:
a) a portable device comprising at least one sensor specific for at least one marker;
b) a prescribed medication comprising a first marker of the at least one marker, wherein said first marker is detectable in bodily fluids and is representative of the prescribed medication; and
c) a central computer that processes information provided by the portable device.

2. The system of claim 1, wherein the central computer comprises a first processing means that performs at least one of the following functions: tracks dispensed prescription medication and corresponding prescription regimen; tracks the markers that are detectable by the portable device; and tracks information regarding the origin of the prescribed medication.

3. The system of claim 2, wherein the first processing means further comprises at least one of the items selected from the group consisting of a means for receiving data provided via the at least one sensor; a means for determining whether an action has occurred within a configurable time interval; a memory device; a data filter; a built-in algorithm; an event indicator; and an artificial neural network.

4. The system of claim 1, wherein the central computer is either directly or remotely connected to the portable device.

5. The system of claim 1, further comprising a communication device that can transfer data from the portable device to the central computer.

6. The system of claim 5, wherein the communication device is selected from the group consisting of: wireless interfaces, cable modems, satellite links, microwave relays, and telephonic modems.

7. The system of claim 5, wherein the communication device permits two-way communication between the portable device and the central computer.

8. The system of claim 1, wherein the sensor is selected from the group consisting of surface acoustic wave sensors; quartz microbalance sensors; metal oxide sensors; bulk acoustic wave sensors; plate acoustic wave sensors; interdigitated microelectrode sensors; optical waveguide sensors; electrochemical sensors; electrically conducting sensors; artificial noses; electronic noses; electronic tongues; semiconductive gas sensors; mass spectrometers; IR, UV, visible, or fluorescence spectrophotometers; apparatuses having conductive-polymer gas-fluorescence spectrophotometers; sensors having conductive-polymer gas sensors; aptamer-based biosensors; ion mobility spectrometry; photo-ionization detectors; amplifying fluorescent polymer sensors; ion mobility spectrometry; thickness-shear mode sensors; microgravimetric sensors; and microcantilever sensors.

9. The system of claim 1, wherein the portable device further comprises a sampling means for providing a sample of the patient's bodily fluid to the sensor of the portable device.

10. The system of claim 9, wherein the sampling means is selected from the group consisting of a wand, chamber, dish, plate, well assay, sheet, film, and dipstick.

11. The system of claim 1, wherein the bodily fluid is selected from the group consisting of exhaled breath, whole blood, blood plasma, urine, semen, saliva, lymph fluid, meningal fluid, amniotic fluid, glandular fluid, vaginal fluid, sputum, feces, sweat, mucuous, and cerebrospinal fluid.

12. The system of claim 11, wherein the bodily fluid is exhaled breath.

13. The system of claim 1, wherein the at least one marker is selected from the group consisting of the prescribed medication; metabolites of the medication; endogenous by-products produced in metabolizing the medication; GRAS additives; and olfactory markers.

14. The system of claim 13, wherein the GRAS additives are selected from the group consisting of dibenzyl ether; difurfuryl ether; ethylene glycol monobutyl ether; furfuryl methyl ether; isoeugenyl benzyl ether; isoeugenyl ethyl ether; isoeugenyl methyl ether; methyl phenethyl ether; beta-naphthyl isobutyl ether; vanillyl butyl ether; dimethylethanolamine; isopentylideneisopentylamine; sodium bisulfate; dioctyl sodium sulfosuccinate; polyglycerol polyricinoleic acid; calcium casein peptone-calcium phosphate; botanicals; ferrous bisglycinate chelate; seaweed-derived calcium; docosahexaenoic acid-rich single cell oil; arachidonic acid-rich single-cell oil; frucooligosaccharide; trehalose; gamma cyclodextrin; phytosterol esters; gum arabic; potassium bisulfate; stearyl alcohol; erythritol; D-tagatose; and mycoprotein.

15. The system of claim 14, wherein the botanicals are selected from the group consisting of chrysanthemum; licorice; jellywort; honeysuckle; lophatherum; mulberry leaf; frangipani; selfheal; and sophora flower bud.

16. The system of claim 13, wherein the olfactory markers are selected from the group consisting of dimethyl sulfoxide; acetaldehyde; acetophenone; trans-Anethole (1-methoxy-4-propenyl benzene) (anise); benzaldehyde (benzoic aldehyde); benzyl alcohol; benzyl cinnamate; cadinene; camphene; camphor; cinnamaldehyde (3-phenylpropenal); garlic; citronellal; cresol; cyclohexane; eucalyptol; eugenol, eugenyl methyl ether; butyl isobutyrate (n-butyl 2, methyl propanoate) (pineapple); citral (2-trans-3,7-dimethyl-2,6-actadiene-1-al); menthol (1-methyl-4-isopropylcyclohexane-3-ol); and α-Pinene (2,6,6-trimethylbicyclo-(3,1,1)-2-heptene).

17. The system of claim 13, where the first marker is the prescribed medication, the prescribed medication further comprising compounds that enhance detection of the markers by the sensor of the portable device.

18. The system of claim 1, wherein the prescribed medication is selected from the group consisting of narcotic analgesics; narcotic analgesics in combination with other medications; medications to treat various mental conditions or disorders; medications for treating erectile dysfunction, medications for treating weight problems; cerebral nervous system depressants; medications to treat diarrhea; and stimulants.

19. The system of claim 18, wherein the prescribed medication is selected from the group consisting of Darvon, Demerol, Dilaudid, Fentanyl, Methadose, MSIR, Nubain, Oxycontin, Roxanol, Stadol, Vicodin; Lorcet; Tylox; Percocet, Diazepam, Paroxetine, Sertraline, Fluoxetine, Viagra, Mebaral, Nembutal, Librium, Xanax, Halcion, ProSom, Adderal; Dexedrine, Ritalin; Focalin; and Provigil.

20. The system of claim 1, wherein the portable device further comprises a second processing means for processing the signals generated by the sensor.

21. The system of claim 20, wherein the second processing means further comprises at least one of the items selected from the group consisting of a means for receiving data provided via the at least one sensor; a means for determining whether an action has occurred within a configurable time interval; a memory device; a data filter; a built-in algorithm; an event indicator; and an artificial neural network.

22. The system of claim 21, wherein the second processing means executes program codes that coordinate various operations of the portable device.

23. The system of claim 22, wherein at least one of the program codes are selected from the group consisting of interaction software; analysis of bodily fluid. software; and calendar software.

24. The system of claim 1, wherein the portable device further comprises an identification system.

25. The system of claim 24, wherein the identification system is selected from the group consisting of a biometric identification system, an electronic coding system, and a lock-and-key identification system.

26. The system of claim 1, wherein the portable device further comprises at least one of the items selected from the group consisting of an input means and a display means.

27. The system of claim 1, wherein the items are selected from the group consisting of an input device; an interactive graphical monitor; a liquid crystal display; and a monitor.

28. The system of claim 1, wherein the portable device is designed using modular sections.

29. The system of claim 1, further comprising a medication container for said prescribed medication, the medication container comprising a second marker, wherein said second marker is indicative of the origins of the prescribed medication.

30. The system of claim 29, wherein said second marker is detectable in the headspace of the container, on a component of said container, or on a packaging item of the container.

31. A method for monitoring patient compliance in taking a medication in accordance with a prescription regimen and tracking dispensed medication comprising:
a) providing information regarding a prescribed medication to a portable device and central computer;
b) distributing the prescribed medication to a patient;
c) exposing a sample of the patient's bodily fluid to at least one sensor of the portable device; and
d) recording and analyzing data from the portable device,
wherein the at least one sensor is specific for at least one marker, wherein the prescribed medication comprises a first marker of the at least one marker, wherein said first marker is detectable in bodily fluids and is representative of the prescribed medication, and wherein said information comprises at least one item selected from the group consisting of: information regarding the prescription regimen; information regarding the first marker; and information regarding the side effects of the prescription medication.

32. The method of claim 31, wherein information regarding the prescription regimen; information regarding the first marker; and information regarding the side effects of the prescription medication are provided to the central computer, wherein the central computer comprises a first processing means that performs at least one of the following functions: tracks dispensed prescription medication and corresponding prescription regimen; and tracks the markers that are detectable by the portable device.

33. The method of claim 32, wherein the first processing means further comprises at least one of the items selected from the group consisting of a means for receiving data provided via the at least one sensor; a means for determining whether an action has occurred within a configurable time interval; a memory device; a data filter; a built-in algorithm; an event indicator; and an artificial neural network.

34. The method of claim 31, wherein the central computer is either directly or remotely connected to the portable device.

35. The method of claim 31, wherein the central computer further comprises a communication device that can transfer data from the portable device to the central computer.

36. The method of claim 35, wherein the communication device is selected from the group consisting of: wireless interfaces, cable modems, satellite links, microwave relays, and telephonic modems.

37. The method of claim 35, wherein the communication device permits two-way communication between the portable device and the central computer.

38. The method of claim 31, wherein the sensor is selected from the group consisting of surface acoustic wave sensors; quartz microbalance sensors; metal oxide sensors; bulk acoustic wave sensors; plate acoustic wave sensors; interdigitated microelectrode sensors; optical waveguide sensors; electrochemical sensors; electrically conducting sensors; artificial noses; electronic noses; electronic tongues; semiconductive gas sensors; mass spectrometers; IR, UV, visible, or fluorescence spectrophotometers; apparatuses having conductive-polymer gas-fluorescence spectrophotometers; sensors having conductive-polymer gas sensors; aptamer biosensors; ion mobility spectrometry; photo-ionization detectors; amplifying fluorescent polymer sensors; ion mobility spectrometry; thickness-shear mode sensors; microgravimetric sensors; and microcantilever sensors.

39. The method of claim 31, wherein the portable device further comprises a sampling means for exposing the sample of the patient's bodily fluid to the sensor of the portable device.

40. The method of claim 39, wherein the sampling means is selected from the group consisting of a wand, chamber, dish, plate, well assay, sheet, film, and dipstick.

41. The method of claim 31, wherein the bodily fluid sample is selected from the group consisting of exhaled breath, whole blood, blood plasma, urine, semen, saliva, lymph fluid, meningal fluid, amniotic fluid, glandular fluid, vaginal fluid, sputum, feces, sweat, mucuous, and cerebrospinal fluid.

42. The method of claim 41, wherein the bodily fluid is exhaled breath and further comprising the steps of: administering the prescribed medication to the patient; after administration of the medication, and providing the sample of exhaled breath to the at least one sensor of the portable device.

43. The method of claim 31, wherein the at least one marker is selected from the group consisting of the prescribed medication; metabolites of the medication; endogenous by-products produced in metabolizing the medication; GRAS additives; and olfactory markers.

44. The method of claim 43, wherein the GRAS additives are selected from the group consisting of dibenzyl ether; difurfuryl ether; ethylene glycol monobutyl ether; furfuryl methyl ether; isoeugenyl benzyl ether; isoeugenyl ethyl ether; isoeugenyl methyl ether; methyl phenethyl ether; beta-naphthyl isobutyl ether; vanillyl butyl ether; dimethylethanolamine; isopentylideneisopentylamine; sodium bisulfate; dioctyl sodium sulfosuccinate; polyglycerol polyricinoleic acid; calcium casein peptone-calcium phosphate; botanicals; ferrous bisglycinate chelate; seaweed-derived calcium; docosahexaenoic acid-rich single cell oil; arachidonic acid-rich single-cell oil; frucooligosaccharide; trehalose; gamma cyclodextrin; phytosterol esters; gum arabic; potassium bisulfate; stearyl alcohol; erythritol; D-tagatose; and mycoprotein.

45. The method of claim 44, wherein the botanicals are selected from the group consisting of chrysanthemum; licorice; jellywort; honeysuckle; lophatherum; mulberry leaf; frangipani; selfheal; and sophora flower bud.

46. The method of claim 43, wherein the olfactory markers are selected from the group consisting of dimethyl sulfoxide; acetaldehyde; acetophenone; trans-Anethole (1-methoxy-4-propenyl benzene) (anise); benzaldehyde (benzoic aldehyde); benzyl alcohol; benzyl cinnamate; cadinene; camphene; camphor; cinnamaldehyde (3-phenylpropenal); garlic; citronellal; cresol; cyclohexane; eucalyptol; eugenol, eugenyl methyl ether; butyl isobutyrate (n-butyl 2, methyl propanoate) (pineapple); citral (2-trans-3,7-dimethyl-2,6-actadiene-1-al); menthol (1-methyl-4-isopropylcyclohexane-3-ol); and α-Pinene (2,6,6-trimethylbicyclo-(3,1,1)-2-heptene).

47. The method of claim 43, where the first marker is the prescribed medication, the prescribed medication further comprising compounds that enhance detection of the markers by the sensor of the portable device.

48. The method of claim 31, wherein the prescribed medication is selected from the group consisting of narcotic analgesics; narcotic analgesics in combination with other medications; medications to treat various mental conditions or disorders; medications for treating erectile dysfunction, medications for treating weight problems; cerebral nervous system depressants; medications to treat diarrhea; and stimulants.

49. The method of claim 48, wherein the prescribed medication is selected from the group consisting of Darvon, Demerol, Dilaudid, Fentanyl, Methadose, MSIR, Nubain, Oxycontin, Roxanol, Stadol, Vicodin; Lorcet; Tylox; Percocet, Diazepam, Paroxetine, Sertraline, Fluoxetine, Viagra, Mebaral, Nembutal, Librium, Xanax, Halcion, ProSom, Adderal; Dexedrine, Ritalin; Focalin; and Provigil.

50. The method of claim 31, wherein the portable device further comprises a second processing means for processing the signals generated by the sensor.

51. The method of claim 50, wherein the second processing means further comprises at least one of the items selected from the group consisting of a means for receiving data provided via the at least one sensor; a means for determining whether an action has occurred within a configurable time interval; a memory device; a data filter; a built-in algorithm; an event indicator; and an artificial neural network.

52. The method of claim 51, wherein the second processing means executes program codes that coordinate various operations of the portable device.

53. The method of claim 52, wherein at least one of the program codes are selected from the group consisting of interaction software; analysis of bodily fluid software; and calendar software.

54. The method of claim 31, wherein the portable device further comprises an identification system.

55. The method of claim 54, wherein the identification system is selected from the group consisting of a biometric identification system, an electronic coding system, and a lock-and-key identification system.

56. The method of claim 55, wherein the identification system is the biometric identification system, and further comprising the step of supplying a biometric feature to the biometric identification system prior to exposing the sample of patient bodily fluid to the at least one sensor of the portable device.

56. The method of claim 31, wherein the portable device is designed using modular sections.

57. A method for determining the origin of a prescription medication comprising:
a) providing information regarding a prescribed medication's origin to a portable device and central computer;
b) exposing an area of a medication container to at least one sensor of the portable device;
d) recording and analyzing data from the portable device,
wherein the at least one sensor is specific for at least one marker, wherein the medication container comprises a first marker of the at least one marker, wherein said first marker is representative of the prescribed medication's origin, and wherein said information comprises information regarding the first marker.

58. The method of claim 57, wherein the area of the medication container is selected from the group consisting of: a headspace, a cap, and a packaging item.

59. The method of claim 57, wherein information regarding the information regarding the first marker is provided to the central computer, wherein the central computer comprises a first processing means that tracks the information regarding the origin of the prescribed medication.

60. The method of claim 57, wherein the central computer is either directly or remotely connected to the portable device.

61. The method of claim 57, wherein the central computer further comprises a communication device that enables communication to the central computer.

62. The method of claim 61, wherein the communication device is selected from the group consisting of: wireless interfaces, cable modems, satellite links, microwave relays, and telephonic modems.

63. The method of claim 61, wherein the communication device permits two-way communication between the portable device and the central computer.

64. The method of claim 57, wherein the sensor is selected from the group consisting of surface acoustic wave sensors; quartz microbalance sensors; metal oxide sensors; bulk acoustic wave sensors; plate acoustic wave sensors; interdigitated microelectrode sensors; optical waveguide sensors; electrochemical sensors; electrically conducting sensors; artificial noses; electronic noses; electronic tongues; semiconductive gas sensors; mass spectrometers; IR, UV, visible, or fluorescence spectrophotometers; apparatuses having conductive-polymer gas-fluorescence spectrophotometers; sensors having conductive-polymer gas sensors; aptamer biosensors; ion mobility spectrometry; photo-ionization detectors; amplifying fluorescent polymer sensors; ion mobility spectrometry; thickness-shear mode sensors; microgravimetric sensors; and microcantilever sensors.

65. The method of claim 57, wherein the at least one marker is selected from the group consisting of the prescribed medication; GRAS additives; and olfactory markers.

66. The method of claim 65, wherein the GRAS additives are selected from the group consisting of dibenzyl ether; difurfuryl ether; ethylene glycol monobutyl ether; furfuryl methyl ether; isoeugenyl benzyl ether; isoeugenyl ethyl ether; isoeugenyl methyl ether; methyl phenethyl ether; beta-naphthyl isobutyl ether; vanillyl butyl ether; dimethylethanolamine; isopentylideneisopentylamine; sodium bisulfate; dioctyl sodium sulfosuccinate; polyglycerol polyricinoleic acid; calcium casein peptone-calcium phosphate; botaicals; ferrous bisglycinate chelate; seaweed-derived calcium; docosahexaenoic acid-rich single cell oil; arachidonic acid-rich single-cell oil; frucooligosaccharide; trehalose; gamma cyclodextrin; phytosterol esters; gum arabic; potassium bisulfate; stearyl alcohol; erythritol; D-tagatose; and mycoprotein.

67. The method of claim 66, wherein the botanicals are selected from the group consisting of chrysanthemum; licorice; jellywort; honeysuckle; lophatherum; mulberry leaf; frangipani; self-heal; and sophora flower bud.

68. The method of claim 65, wherein the olfactory markers are selected from the group consisting of dimethyl sulfoxide; acetaldehyde; acetophenone; trans-Anethole (1-methoxy-4-propenyl benzene) (anise); benzaldehyde (benzoic aldehyde); benzyl alcohol; benzyl cinnamate; cadinene; camphene; camphor; cinnamaldehyde (3-phenylpropenal); garlic; citronellal; cresol; cyclohexane; eucalyptol; eugenol, eugenyl methyl ether; butyl isobutyrate (n-butyl 2, methyl propanoate) (pineapple); citral (2-trans-3,7-dimethyl-2,6-actadiene-1-al); menthol (1-methyl-4-isopropylcyclohexane-3-ol); and α-Pinene (2,6,6-trimethylbicyclo-(3,1,1)-2-heptene).

69. The method of claim 57, wherein the portable device further comprises a second processing means for processing the signals generated by the sensor.

70. The method of claim 69, wherein the second processing means further comprises at least one of the items selected from the group consisting of a means for receiving data provided via the at least one sensor; a means for determining whether an action has occurred within a configurable time interval; a memory device; a data filter; a built-in algorithm, an event indicator; and an artificial neural network.

71. The method of claim 69, wherein the second processing means executes program codes that coordinate various operations of the portable device.

72. The method of claim 71, wherein at least one of the program codes are selected from the group consisting of interaction software; analysis of the marker software; and calendar software.

73. The method of claim 57, wherein the portable device is designed using modular sections.
